(19) United States
(12) Patent Application Publication
(10) Pub. No.: US 2009/0047313 A1
(43) Pub. Date: Feb. 19, 2009

(54) PREPARATION HAVING SKIN-MOISTURIZING PROPERTIES

(75) Inventors: Werner Bruechert, Oberasbach (DE); Irina Daichendt, Nuernberg (DE); Willy Weiss, Altdorf (DE)

Correspondence Address: BACHMAN & LAPOINTE, P.C., 900 CHAPEL STREET, SUITE 1201, NEW HAVEN, CT, 06510, US

(73) Assignee: SCHWAN-STABILO COSMETICS GMBH & CO. KG, Heroldsberg (DE)

(21) Appl. No.: 12/193,078

(22) Filed: Aug. 18, 2008

(30) Foreign Application Priority Data
Aug. 17, 2007 (DE) 20 2007 011 568.7

(51) Int. Cl. A61K 8/00; A61K 8/34; A61K 8/72; A61Q 1/06; A61Q 1/10

(52) U.S. Cl. 424/401; 424/70.11; 424/63; 424/64

(57) ABSTRACT

A permanently skin-moisturizing cosmetic preparation and use thereof.

### FIELD OF THE INVENTION

The invention concerns a cosmetic preparation having skin-moisturizing properties.

### BACKGROUND OF THE INVENTION

There has always already been a wish on the part of human beings to give a more youthful appearance to their skin. Beauty and therewith also perfection of the skin has always been an important criterion for standing out from the crowd. While previously simple household agents comprising vegetable extracts combined with selected oils were often used, nowadays dedicated active substance complexes are offered for any skin type and requirement, in order to care for the skin and to improve its structural image.

Inundated with the large number of active substances which are available on the market and which often have an exotic sound to them, the trend in recent times is to go back again to the well-tried, simple and natural care substances. Modern cosmetics therefore once again contain fats with a care action such as shea butter or coconut oil, extracts from chamomile or marigold and moisture-retaining agents such as sorbitol or glycerin. In particular glycerin has recently been enjoying a revival.

Glycerin has already long been known for use in cosmetics. Various skin creams are advertised with the care properties inherent in glycerin. Glycerin is a trivalent alcohol with a slightly sweetish taste, which dissolves very well in water by virtue of its hydrophilic properties. Equally its skin-moisturizing effect is also attributed to its hydrophilia as it is thereby capable of binding water out of the environment and transporting it into the skin. The cells of the skin are thus supplied with additional water whereby they increase in pressure and thus volume. The result is a taut, smooth skin.

In particular creams such as hand creams and face care creams contain glycerin for regulating the moisture content of the skin.

Because of the increasing stress placed on the skin due to environmental influences such as increased UV radiation and single-sided feeding, as well as insufficiently humidified heating air in office premises, the skin is often very dry, cracked and irritated and tightens. As a result the demands on the respective cosmetics which are intended to neutralize those effects increase. Accordingly in the case of glycerin which serves as a skin moisturizing agent high levels of concentration, over 10% by weight with respect to the total weight of the mass, are desirable. In addition the desire for moisture-contributing cosmetics occurs not only in the purely care sector but increasingly also for decorative cosmetics.

DE 699 11 489 discloses cosmetic preparations with up to 10% by weight of glycerin for the treatment of skin diseases (for example rosacea). A lipstick is also available on the market (Novalip Lissage extreme) which contains 8% by weight of glycerin. As however a content of 8% by weight of glycerin is a skin portion and in particular cannot permanently moisturize the lips, the wish for cosmetics with higher contents of glycerin, with at least between about 10 and 50% by weight, is consistently high.

Hitherto however it was difficult if not entirely impossible to stabilize cosmetic materials with a high proportion of glycerin, in particular if this involved water-reduced or entirely water-free cosmetics. In other words, it was hitherto not possible to provide impressive storable products with more than about 10% by weight of glycerin in the decorative field.

As already stated glycerin is a hydrophilic, water-soluble substance. Current, glycerin-bearing cosmetics therefore generally include a large proportion of water as a suitable solvent for the trivalent alcohol. The result of this is that those cosmetics are those of emulsion type, in particular O/W emulsions are to be found here. However, even in the case of water-bearing cosmetics of emulsion type, stable formulation of high levels of concentration of glycerin is often difficult as interactions can occur with thickeners contained in the preparation. The fact that the glycerin molecules are homogeneously distributed among the water molecules means that the thickener cannot comprehensively produce its gelling action on the water molecules. The consequence is unstable emulsions which with time have a tendency to undergo separation.

Cream jars or tubes are highly suitable for long-term stable storage of even sensitive materials for they are subjected to a filling procedure which is less problematical for the material and even when the material is removed therefrom by the consumer, mostly by dipping a finger into the material, or by simply allowing it to run out, as in the case of lotions, the material is scarcely subjected to mechanical stresses. As however they are unwieldy and unsuitable for use while away, there is a wish for more easily manageable products. Those in pencil form are particularly suitable for that purpose. The production and further processing of cosmetic materials for use in a cosmetic pencil is in that respect however markedly more difficult than in the case of cream and lotion products.

A serious disadvantage of water-bearing cosmetics is their susceptibility to germ growth as water represents a medium which is suitable for the proliferation of germs. In addition emulsions are far from being as stable in respect of storage as single-phase systems, particularly at higher temperatures, with the result that the cosmetics break down and therefore quickly become unusable. There is therefore a low level of acceptance by the consumer. In addition, due to the high vapor pressure of the water, high demands are made in terms of sealing integrity of the system, that is to say the application medium. Furthermore emulsions contain emulsifiers and surface-active substances for stabilization thereof. Among those, in particular highly ethoxylated compounds are preferred as they are simple to incorporate and impart adequate stability to the material. Emulsifiers in general and ethoxylated compounds in particular however involve an enormous irritation potential. Acceptance of such products by the consumer is therefore limited. There is thus increasingly a wish to achieve a skin-moisturizing action in respect of glycerin even in water-free cosmetics in stable, highly glycerin-bearing preparations.

Therefore an object of the invention is to provide a cosmetic preparation for the care of or improvement in the skin, which can lastingly moisturize the skin.

A further object of the invention is to provide a skin-care and durably skin-moisturizing cosmetic preparation which can be processed to afford a mechanically loadable stick or refill.

Furthermore an object of the invention is that of providing a preparation which can give sticks or refills which have not only mechanical stability but which at the same time also have a good application profile. The preparation is to be soft, homogeneous and pleasant to apply, it is to adhere after application to the location involved, that is to say it is not to transfer or is to transfer only very slightly to articles which come into contact therewith, when applied to the eyes or lips it is not to migrate into the wrinkles, it is not to smudge or smear and in addition it is to be very substantially water-resistant. A further demand on the cosmetic preparation is its storage stability. Both at high temperatures and also at very low temperatures, as are usual in the various countries in the world during the different seasons of the year, the preparation is not to perceptibly alter its consistency and thus its application properties, nor is it to have optical separation effects such as syneresis, creaming, drying out or the like.

Furthermore the preparation according to the invention is to be microbiologically stable. Microbiologically stable signifies here that the growth of germs and in particular the growth of pathogenic germs is substantially suppressed.

### SUMMARY OF THE INVENTION

Those objects are attained by a preparation where it has been surprisingly found that a combination of glycerin and polyglyceryl-10 behenate/eicosadionate provides a preparation which imparts tone to the skin but at the same time has such mechanical properties that it can be processed to constitute mechanically loadable parts such as sticks or refills. Specific features and embodiments are set forth in the appendant claims. Unless otherwise specified all percentages relate to weight and all weight units (% by weight) relate to the total weight of the material.

Mechanical stability in connection with the preparation according to the invention signifies that, even in the long term, at different temperatures and transport conditions, such a preparation does not separate or experience syneresis, and even at varying temperatures it does not alter its physical properties noticeably for the consumer. Mechanical stability in the case of the preparation according to the invention further signifies that the preparation can be put into the form of a structured material which retains its shape and also its stability upon application under the influence of pressure and shearing force.

The area of application of the preparation according to the invention is not limited in terms of location. The preparation can be applied both to the entire surface of the skin and also the mucous membranes and the semi-mucous membranes. If hereinafter application to the skin is described, that is also intended to include mucous membranes and semi-mucous membranes. After application during the period for which it is worn the cosmetic preparation does not lead to tautness effects and it does not dry out the applied surfaces. Rather, the preparation according to the invention provides that the surfaces to which it is applied are durably moisturized and as a result become supple so that in terms of overall impression they give a taut, youthful appearance.

Furthermore the cosmetic preparation according to the invention with durably skin-moisturizing properties can be shaped to provide sticks or refills which can then be used in many different forms, they can be both glued into wood and cast in cases and can also be inserted into a rotary mechanism. In particular the stability of the cosmetic preparation according to the invention is so great that it can be used as a freestanding stick.

By virtue of its advantageous properties the cosmetic preparation according to the invention can be used for long-lasting moisturizing of the skin as a concealer pencil and in addition can be used as eyeliner, eyeshadow or lipliner. In a particularly preferred embodiment the preparation according to the invention is in the form of a lipstick.

### DETAILED DESCRIPTION

What is essential for the good properties of the preparation according to the invention is the formation of a specific structure which comes about by virtue of the combination of glycerin and polyglyceryl-10 behenate/eicosadionate. For the production of cosmetic pencils, it is necessary for the cosmetic preparation to afford the capability of being able to produce a certain structure. The reference to forming the structure of a cosmetic material is used to denote the capability on the part of the mixture of raw materials, from which the cosmetic material is made up, to solidify. As in the case of aqueous preparations, that can occur by gelling and formation of a gel by virtue of an added thickener or, as in the case of both emulsions and also purely lipophilic systems, by consolidation of the structure-imparting waxes and fats. The appropriate combination of raw materials is decisive for the later properties and thus also the stability of the product formed. The mixture of raw materials according to the invention affords a structure-forming material which can be shaped to provide mechanical loadable sticks or refills.

Sticks or refills are shaped in per se known manner generally either while still in the fluid state by casting of the material or in the state of already having cooled down by extrusion thereof. A material which is shaped by extrusion to provide sticks can be for example glued into wood or can be fitted into a device in the form of a freestanding stick or refill. Another possibility involves casting the material while in the still fluid heated condition in suitable cases, generally plastic material cases. The cosmetic material then hardens in the cases to give a structured material in stick form. As the cases in which the cosmetic material is cast are held in a conical pouring mask in order to shape the conical tips which are usually desired so that the liquid material flows into the pouring mask through the pencil casing and the completed pencil shape is formed there after hardening, it is important that the tips can be properly removed from the casting molds without remaining stuck thereto or breaking. The pencils which are formed in that way and which contain the structured material in stick or refill form are products which can be formed to a point and which can be offered both as liner products for the eye area or the lip area and also products which can be applied over a surface such as lipstick, concealer pencil or eyeshadow.

In addition however it is also possible to insert the structured material which is in a cold condition and which is extruded to put it into the form of a stick or pencil into a rotary mechanism or the heated material while still fluid can also be cast directly into a suitable cartridge which embraces the rotary mechanism. In the former case the result is a product with a freestanding stick while in the second case the stick bears against the inside wall of the cartridge.

If the cosmetic, commercially available different products are considered side-by-side, it is to be noted that the demands made on a material which is processed to afford sticks in order to form a cosmetic pencil are high. The materials must be suitable for forming stable sticks therefrom, and in particular the mechanical stability of the sticks is of interest in that respect.

The reference to mechanical stability of cosmetic sticks or refills means that the sticks or refills do not suffer any damage in daily use, more specifically neither by application of the stick or pencil which contains the stick, nor by virtue of the different storage conditions acting on the pencil, inter alia therefore high/low temperatures, shaking movements when being transported and also falling from relatively great heights. More specifically when a cosmetic pencil is applied to the skin extremely high shearing forces act on the stick thereof perpendicularly to the longitudinal axis of the stick, in particular in the front region, the tip thereof. The pressure loading when the pencil is moved sideways on the skin can result in the tip of the pencil breaking off at the conical end of the stick case, in the event of inadequately stabilized materials. The pencil becomes useless as a result and has to be re-sharpened or, in the case of pencils with a rotary mechanism, further extended by rotation of the mechanism. The pressure and shearing loading on the stick is extremely high in particular in those pencils which have a stick of such a nature that its diameter is very small in comparison with its length. That is the case with liner products.

Even more than in the case of sticks or refills fitted in a case, freestanding sticks which are made from an inadequately stable material suffer from the risk of the stick breaking off, mostly in the rear region of the stick, more precisely where the stick holder ends. For, in the region where the material is anchored in the stick holder, it is held in a stable condition by the wall of the holder, but in contrast the portion projecting therefrom is not laterally stabilized. Shearing forces and pressures as are applied when the pencil is applied to the skin lead to tilting of the unstabilized stick portion whereby the stick breaks and as a consequence slips out of the surrounding cartridge or case. The preparation according to the invention now provides sufficiently stable materials to overcome that problem.

In summary it can be established that the cosmetic materials according to the invention, by virtue of the specific combination of glycerin and polyglyceryl-10 behenate/eicosadionate have the capability of producing a stable structure which homogeneously contains all raw materials of the matrix so as to prevent the formation of desired-rupture locations in manufacture of the cosmetic material.

The cosmetic preparation according to the invention provides in particular pencils for moisturizing of and caring for the skin and dry portions of the skin and also makes it possible to afford the positive care characteristics of glycerin in cosmetic pencils since, as already mentioned, by virtue of their limited dimensions, such pencils are simple to keep in a handbag or pants pocket so that they can be applied while out and about.

Glycerin, that is to say propane triol, can be easily incorporated into aqueous preparations but that becomes difficult if the content of glycerin rises above 10% and in particular if the product to be manufactured therefrom is to become a structured material in stick or refill form. Emulsions in stick form are less stable. By virtue of the nature of the emulsion, that is to say distribution of droplets of one phase in another, the network between the components is labile and has a tendency to break or smear upon the application of a shearing force as is applied when the material is applied to the skin. The mechanical stability of emulsions in pencil form is thus limited. In particular the incorporation of high contents of glycerin into such a stick emulsion is still much more difficult since, as already stated, glycerin has an additional destabilizing effect by virtue of disturbing the gel network produced by the thickener. The implementation of preparations with a high glycerin content in stick form has therefore hitherto not been possible. That also applies to water-free formulations in stick form. It will be noted however that here the repulsive interactions of the hydrophilic glycerin with the lipophilic matrix contribute decisively to destabilization of the system.

As already stated the invention provides a cosmetic preparation for care and appearance improvement purposes. The skin is lastingly moisturized by use of the preparation according to the invention. To achieve that effect the preparation according to the invention includes a high proportion of the caring and skin-moisturizing substance glycerin, that is to say propane triol. The reference to a high proportion of glycerin means a proportion with between more than 10% by weight and about 50% by weight with respect to the total weight of the material. As already stated lower contents than 10% by weight do not lead to the desired, long-lasting and sufficient moisturizing of the part of the skin to which the preparation has been applied. Admittedly, contents of higher than 50% by weight still have a very good moisturising effect but the application of such a preparation can be perceived as being scratchy and sandy and therefore no longer pleasant. In addition such preparations can give the feel of being sticky. Particularly good application properties and also results which are optimum in respect of the moisturizing characteristic can be achieved if a content of between about 15 and 35% by weight of glycerin is incorporated into the cosmetic preparation. This involves a balanced relationship in terms of very good moisturizing effect and pleasant application even during a prolonged period of wearing the preparation.

Besides its caring and skin-moisturizing properties however the preparation according to the invention also has the desired stability. That is achieved by the combination of the glycerin with a polyglycerylester, namely polyglyceryl-10 behenate/eicosadionate.

Countless glycerin derivatives are available on the market. The use of glyceryl derivatives such as glyceryl esters for the stabilization of hydrophilic substances in a predominantly lipophilic environment has long been known. In particular the mono- and diesters of glycerin also serve as emulsifiers and are therefore used in emulsions. It was surprisingly found that polyglyceryl-10 behenate/eicosadionate is most suitable for stabilizing high contents of glycerin, that is to say contents of between about 10 and 50% by weight. The combination of glycerin and polyglyceryl-10 behenate/eicosadionate is suitable both for use in emulsion-based preparations and also in water-free, lipid-based systems. As it does not bear any long ethoxylated side chains it is extremely skin-friendly and does not lead to irritations, even when frequently used.

In that respect the term water-free systems is used to denote those to which no water was added as an independent raw material. By virtue of hygroscopic effects however traces of water can be introduced into the formulation, in particular due to the possible presence of pigments or fillers or other substances.

Without being bound down to a theory, the raw material polyglyceryl-10 behenate/eicosadionate appears to involve such a balanced ratio of lipophilic to hydrophilic components that even amounts of up to 50% by weight of glycerin in an otherwise lipophilic base can be stabilized without any problem. Due to the behenyl and eicosane groups, the raw material is very well incorporated into the lipophilic structure, in which respect the inventors established that a polyglyceryl unit of 10 glycerin residues is optimum to introduce glycerin in such large amounts into the lipophilic matrix without syneresis effects or other destabilizing effects occurring in the long term even at high temperatures. Cosmetic preparations which contain a combination of the raw materials glycerin and polyglyceryl-10 behenate/eicosadionate are stable in storage over a number of weeks at 50° C. and do not perceptibly alter their consistency and thus their application properties. Polyglyceryl-10 behenate/eicosadionate is therefore suitable both for the production of emulsions with high contents of glycerin and also corresponding water-free purely lipophilic systems.

The polyglyceryl-10 behenate/eicosadionate is particularly suitable for the production of cosmetic preparations with a high glycerin content in stick form. Due to its melting point of about 68° C. it imparts stability to the cosmetic material without making the materials brittle and fragile, as is the case with waxes which melt at a markedly higher temperature. To achieve adequate stabilization of the cosmetic preparations the polyglyceryl-10 behenate/eicosadionate is used with between about 2 and 10% by weight with respect to the content of glycerin. A lower concentration than 2% by weight with respect to the glycerin content does not lead to permanent stabilization of the glycerin in the material so that the material has a tendency to separation phenomena and thus becomes unusable for the user. A higher content of polyglyceryl-10 behenate/eicosadionate admittedly leads to a further increase in stability of the homogeneity of the cosmetic preparation but the material, due to the wax, then becomes markedly firmer and duller. The cosmetic preparation thus imparts its desired light, soft and creamy application. A cosmetic preparation with a particularly pleasant application profile with very good stability for the material is achieved if the polyglyceryl-10 behenate/eicosadionate is incorporated in a proportion of between 3 and 8% by weight, with respect to the proportion of glycerin contained in the material.

The masses of the cosmetic preparation according to the invention can be used to form sticks or refills which can be glued into wood, cast into cases or inserted into a rotary mechanism. The resulting product is a cosmetic pencil which includes the structured material in the form of a stick or refill. The sticks which were formed from the structured material according to the invention can vary greatly in their diameter, depending on the respective area of use, and include a range of diameter of between about 1 and 30 mm, preferably between about 3 and 20 mm and particularly preferably between about 8 and 20 mm. Particularly preferably, pencils which, like commercially available lipsticks, have a freestanding stick or refill therein, can be produced from the material according to the invention. In that respect the diameter of that lipstick refill is preferably in a range of between 8 and 20 mm as that order of magnitude is very highly suited to being applied to the lips over a large area. Smaller diameters of between 3 and 8 mm and even less than 3 mm are suitable in particular for using the structured material as a liner product, that is to say eyeliner or lipliner. Sticks or refills which were made from structured materials of a diameter of greater than 20 mm are best suited for face uses, that is to say concealer materials or blushers or generally colorless care pencils. In that respect the sticks or refills formed from the structured material can be extended by a suitable rotary mechanism.

The sticks or refills produced from the structured cosmetic material can be both held in encased relationship (in wood or in plastic cases) and also in a freestanding condition. While the pencils which contain encased sticks are generally liner products, freestanding sticks or refills are used in particular for the production of lipsticks or blushers, as well as concealer pencils.

The cosmetic preparation can be varied by the man skilled in the art in known fashion for manufacture of the different kinds of pencils. Particular attention however is to be paid to the stability of the preparation, which is decisively controlled by the composition thereof. It is precisely in the case of sticks or refills in which the diameter is extremely small in comparison with its length, as in the case of liner products or however in the case of the freestanding sticks which are often even more delicate, that the combination of the raw materials plays a crucial part.

It was surprisingly found that it is precisely delicate sticks or refills such as liner sticks and in particular freestanding cosmetic sticks, that are markedly stabilized by a combination of the raw material glycerin with polyglyceryl-10 behenate/eicosadionate. In that respect the stabilizing effect is a multiple higher than with other current glyceryl and polyglycerylesters. Without being bound to a theory, as stated above, that could be because of the balanced relationship between lipophilic and hydrophilic components in the polyglyceryl-10 behenate/eicosadionate.

The challenge for the cosmetic developer of pencil cosmetics with freestanding sticks lies in finding the appropriate raw materials and the combination thereof. Added to that, in the case of the present invention, is permanent stabilization of the glycerin in the material which can be both a lipophilic matrix and also an aqueous system.

In addition however an important aspect also lies in manufacture of the cosmetic stick. The materials are generally melted and then transferred into suitable casting molds. After the setting operation which is effected by external cooling the stick is removed from the mold and introduced into a suitable stick holder. So that the materials can be removed from the casting molds without residue they must very rapidly homogeneously harden and may not adhere to the wall.

Glycerin is a raw material which is sticky by virtue of its very nature. High contents of glycerin therefore signify difficulties in removal thereof from the casting mold. In addition the materials with a higher melting range, that is to say materials with waxes and fats with a high melting point (melting range significantly over 80° C.) can be more easily removed from the mold without leaving a residue, as they already severely contract and harden at much lower temperatures. By virtue of those prerequisites it was correspondingly more surprising that it is precisely a combination of high contents of glycerin with polyglyceryl-10 behenate/eicosadionate which has a markedly lower melting range, that in a cosmetic preparation leads to a material which can be easily removed from a mold. That effect was not to be predicted and, without being bound down to a theory, can basically be explained in that, due to the polyglyceryl-10 behenate/eicosadionate, the glycerin is virtually completely embedded into the three-dimensional structure thereof so that it loses its stickiness and solely the lipophilic properties of the behenate and eicosadionate residues remain behind. As, by virtue of their voluminous structure, they are not suitable for cross-linking with other lipophilic molecules, they crystallize out quickly, within a short temperature interval. Cosmetic materials of that kind rapidly contract during the cooling operation in the casting mold and come free from the wall without any residue.

That stabilizing effect is presented both in relation to water-free cosmetic preparations and also in relation to aqueous emulsions and thereamong in particular O/W emulsions. It is precisely in the O/W emulsion area in which the water phase which contains the glycerin forms the outer phase that it is important to suppress the sticky properties of the glycerin to such an extent that it is no longer possible for the pourable material to adhere to the wall of the casting mold. It is only in that way that it is ensured that the casting can be removed from the casting mold without any residue and the stick or refill does not suffer any damage. Due to its lipophilic residues the polyglyceryl-10 behenate/eicosadionate forms around the glycerin a lipophilic layer which can be referred to as a lubricant layer. As the polyglyceryl-10 behenate/eicosadionate can be well homogeneously dispersed in the water phase by virtue of its polyglyceryl residue, that stabilizing effect is to be found along the entire cast stick. The casting can be removed from its mold without any residue. In addition there is a further advantage: the fact that the polyglyceryl-10 behenate/eicosadionate is present in the water phase means that, if the concentration of use is in the preferred range, by virtue of the relatively high melting range of the wax, it is possible to dispense with the use of thickeners for production of structured materials. That saves on raw material costs, reduces potential germ involvement which occurs in particular with vegetable thickeners and imparts to the preparation upon use thereof a substantially creamier feel in application than current gel-forming agents such as xanthan gums, carrageenans or gelatins.

In order further to stabilize the cosmetic preparation with a high content of glycerin, an alkoxylated fatty alcohol can be useful as a further additive, in particular if lipophilic oils are to be incorporated. Tests have shown that glycerin, in particular in such high concentrations as are used in the preparation according to the invention, and also polymeric glycerin derivatives such as polyglyceryl-10 behenate/eicosadionate, are not compatible with lipophilic oils as are usual for caring cosmetics. Such materials have a tendency for separation of the glycerin in the solid state, even if the materials appear homogeneous in the molten state.

In a preferred embodiment therefore an alkoxylated fatty alcohol is added to the preparation. Raw materials such as PPG-3 myristylether, PEG-16 cetyl/oleyl/stearyl/lanolin alcohol ether, di-PPG-3 myristyl ether adipate, PEG/PPG-4/2 propylheptyl ether, PEG/PPG-6/2 propylheptyl ether, polyglyceryl-3 cetyl ether, polyglyceryl-3 decyltetradecyl ether, PPG-10 cetyl ether, PPG-20 cetyl ether, PPG-2 lanolin alcohol ether, PPG-5 lanolin alcohol ether, PPG-4 myristyl ether, PPG-11 stearyl ether and PPG-15 stearyl ether have proven to be particularly suitable as ‘stabilizers’. When used in amounts of between 15 and 35% by weight, they can provide that the polyglyceryl-10 behenate/eicosadionate which in turn stabilizes the glycerin remains fixedly bound in the lipophilic matrix so that the cosmetic material remains homogeneous and thus stable even after cooling. Preferably alkoxylated fatty alcohols as set forth hereinafter are used as stabilizers as they can be particularly well processed:

PPG-3 myristylether, PEG-16 cetyl/oleyl/stearyl/lanolin alcohol ether, PEG/PPG-4/2 propylheptyl ether, PEG/PPG-6/2 propylheptyl ether, PPG-10 cetyl ether, PPG-20 cetyl ether, PPG-2 lanolin alcohol ether, PPG-5 lanolin alcohol ether, PPG-4 myristyl ether, PPG-11 stearyl ether and PPG-15 stearyl ether.

In this case also the ratio of hydrophilic to lipophilic components in the alkoxylated fatty alcohol again appears to be decisive, thereby in fact achieving a virtually emulsifier effect.

Without any problems 5% by weight of current oils such as castor oil, triglycerides or jojoba oil can be incorporated into a material in which such a stabilizing alkoxylated fatty alcohol is incorporated, without involving separation phenomena in the material.

Besides those raw materials the preparation according to the invention can contain further additives such as coloring substances. They include both dyes and also pigments. If hitherto it was only possible to introduce dyes into a lipophilic matrix by surfactants, that is achieved in the preparation according to the invention solely by the high proportion of glycerin. The dyes are dissolved therein, thereby producing shimmering, radiant and permanently coloring products which are particularly suitable for blusher, lipliner and lipsticks.

Just the following are listed by way of example among the pigments, but this listing is in no way restrictive, an extensive choice is best known to the man skilled in the art: carbon black, iron oxides, chromium oxide, chromium hydroxide, iron cyanides, ultramarine, titanium dioxide, zinc oxide, metal powders such as for example copper or aluminum, organic pigments and their lakes or carmine. Pearlescent pigments of any nature are also used.

Fats, waxes, fillers, further hydrophilic substances, care substances, flavoring substances, perfumes, anti-oxidants, preserving agents and others are included as further additives to the preparation according to the invention.

The cosmetic preparation produced therefrom can serve both for the care of and also improving appearance in the sense of colored decoration of the face. In particular the preparation according to the invention is suitable for care of and decoration of the lips.

Therefore the preparation according to the invention can be used as a caring cream or paste, as a lip care pencil, lipstick, lipliner, eyeliner, eyeshadow, blusher and concealer pencil. Use as a lipstick is particularly preferred as in particular the sensitive semi-mucous membranes are often plagued with dryness.

The examples hereinafter are intended to serve to illustrate the invention but in no way to limit it.

### EXAMPLES

### Example 1

### Lipstick

A lipstick was produced from the following components (raw materials identified by the INCI nomenclature; the amounts are specified in percent by weight)

| | |
|---|---|
| Pigmente | 9.95 |
| Fillers | 3.0 |
| Ascorbyl palmitate | 0.05 |
| Glycerin | 30.0 |
| Polyglyceryl-10 behenate/eicosadionate | 3.0 |
| PPG-3 myristylether | 30.0 |
| Cera microcristallina | 14.0 |
| Polyglyceryl-3 polyricinoleate | 5.0 |
| Polybutene | 5.0 |

### Production:

The raw materials were homogenized without pigments and waxes. The waxes and pigments were then added and the composition was heated until the result was a clear homogeneous material. The material was further homogenized on a three-roll mill. Thereafter it was again heated to above its melting point and cast into suitable lipstick molds. After cooling the sticks were removed from the mold and fitted into suitable stick holders.

The lipstick obtained involved soft creamy application, good color transfer and surprisingly had no-transfer properties in relation to ceramic or glass-like articles. After just a short application the lips were cared for, permanently moisturized and gave a fuller, richer impression. Minor lip wrinkles were alleviated and in particular the structure of dry lips was markedly improved. The lipstick was still stable after storage for 3 weeks at 50° C. and exhibited no separation effects.

### Example 2

### Blusher

A blusher was produced from the following components (raw materials identified by the INCI nomenclature; the amounts are specified in percent by weight)

| | |
|---|---|
| Coloring agents | 8.0 |
| Fillers | 2.5 |
| Ascorbyl palmitate | 0.05 |
| Chamomilla Recutita | 0.20 |
| Glycerin | 15.0 |
| Polyglyceryl-10 behenate/eicosadionate | 0.30 |
| PPG-2 lanolin alcohol ether | 25.0 |
| Candelilla | 14.0 |
| Buxus Chinensis Oil | 2.0 |
| Polydecene | 3.0 |
| PVP/eicosene copolymer | 10.0 |
| PEG-6 cetylalcohol | 19.95 |

### Production:

The dyes were dissolved in glycerin. The solution was then added to the remaining raw materials without the waxes and homogenized. The waxes were then added and the composition heated until the result was a clear homogeneous material. The material was rolled at least three times on a three-roll mill and in that case further homogenized. The material after rolling was introduced into cartridges and put into suitable containers.

The blusher obtained could be pleasantly applied to the cheeks with a brush or the fingers. It involved a soft, easy creamy application, it could be well distributed and imparted a natural color to the skin. When the material was drawn into the skin the material was transfer-resistant. The material exhibited no separation phenomena after storage for 3 weeks at 45° C. and could be well applied without change. Particularly upon being applied to dry areas of skin on the cheeks, they were smoothed after prolonged use and appeared markedly less rough.

### Example 3

### Eye Care Pencil

An eye care pencil was produced from the following components (raw materials identified by the INCI nomenclature; the amounts are specified in percent by weight)

| | |
|---|---|
| Parabene | 0.4 |
| Fillers | 12.95 |
| Ascorbyl palmitate | 0.05 |
| Jojoba esters | 15.6 |
| Glycerin | 35.0 |
| Polyglyceryl-10 behenate/eicosadionate | 3.5 |
| PPG-3 myristylether | 16.5 |
| Lanolin | 12.0 |
| Ricinus Communis | 1.0 |
| Isostearyl isostearate | 1.0 |
| Ozocerite | 2.0 |

### Production:

The raw materials were weighed together and homogenized with melting and agitation until the result was a clear homogeneous material. The material was further homogenized on a three-roll mill. Thereupon it was again heated to over its melting point and cast into suitable cases and cooled to room temperature.

The eye care pencil cared for flaky eyelids and could be applied both with the fingers and also directly with the pencil tip to the eyelids. It involved a soft, very gentle sliding application, it could be well distributed and it did not shine. When the material was drawn into the skin it was not sticky and was scarcely perceptible on the eyelids. The pencils did not exhibit any separation phenomena after storage for 4 weeks at 50° C. and could be well used without any change. The dry skin of the eyelids was smoothed, whereby the eyes again acquired a bright appearance.

1. A cosmetic preparation comprises a combination of glycerin and polyglyceryl-10 behenate/eicosadionate, wherein the content of glycerin is at least 10% by weight with respect to the total weight of the preparation.

2. A cosmetic preparation as set forth in claim 1 wherein the preparation is in the form of a structured material.

3. A structured material as set forth in claim 2 wherein the preparation is in the form of a stick which can be glued into wood.

4. A structured material as set forth in claim 2 wherein the preparation is castable and removable from a mold.

5. A structured material as set forth in claim 2 wherein the preparation is in the form of a stick which can be fitted into a rotary mechanism.

6. A structured material as set forth in claim 2 wherein the preparation is in the form of a freestanding stick.

7. A structured material as set forth in claim 1 wherein the preparation is in the form of a stick wherein the diameter of the stick is about 1-30 mm.

8. A structured material as set forth in claim 1 wherein the preparation is in the form of a stick wherein the diameter of the stick is about 3-20 mm.

9. A structured material as set forth in claim 1 wherein the preparation is in the form of a stick wherein the diameter of the stick is about 8-20 mm.

10. A structured material as set forth in claim 1 wherein the proportion of glycerin is between about 10 and 50% by weight with respect to the total material.

11. A structured material as set forth in claim 1 wherein the proportion of glycerin is between about 15 and 35% by weight with respect to the total material.

12. A structured material as set forth in claim 1 wherein the proportion of polyglyceryl-10 behenate/eicosadionate is between about 2 and 10% by weight with respect to the proportion of glycerin.

13. A structured material as set forth in claim 1 wherein the proportion of polyglyceryl-10 behenate/eicosadionate is between about 3 and 8% by weight with respect to the proportion of glycerin.

14. A structured material as set forth in claim 1 wherein the preparation contains at least one alkoxylated fatty alcohol.

15. A structured material as set forth in claim 7 wherein the alkoxylated fatty alcohol is selected from the group consisting of: PPG-3 myristylether, PEG-16 cetyl/oleyl/stearyl/lanolin alcohol ether, PEG/PPG-4/2 propylheptyl ether, PEG/PPG-6/2 propylheptyl ether, PPG-10 cetyl ether, PPG-20 cetyl ether, PPG-2 lanolin alcohol ether, PPG-5 lanolin alcohol ether, PPG-4 myristyl ether, PPG-11 stearyl ether and PPG-15 stearyl ether.

16. A structured material as set forth in claim 15 wherein the proportion of alkoxylated fatty alcohol is between about 15 and 35% by weight with respect to the total material.

17. A structured material as set forth in claim 1 wherein the preparation contains at least one lipophilic oil.

18. A structured material as set forth in claim 17 wherein the proportion of lipophilic oil is up to about 5% by weight with respect to the total material.

19. A structured material as set forth in claim 17 wherein, as a further lipophilic substance, the preparation contains at least one wax.

20. A structured material as set forth in claim 1 wherein the preparation contains coloring substances.

21. A structured material as set forth in claim 19 wherein the preparation it contains at least one soluble dye.

22. A structured material as set forth in claim 19 wherein the preparation contains at least one pigment.

23. A structured material as set forth in claim 1 wherein the preparation contains fats, fillers, hydrophilic substances, care substances, perfumes, flavoring substances, anti-oxidants and preserving agents.

24. A structured material as set forth in claim 1 wherein the preparation is free from water used as an independent raw material.

25. Eyeshadow containing a cosmetic preparation as set forth in claim 1.

26. Eyeliner containing a cosmetic preparation as set forth in claim 1.

27. Lipliner containing a cosmetic preparation as set forth in claim 1.

28. Lipstick containing a cosmetic preparation as set forth in claim 1.

29. Concealer pencil containing a cosmetic preparation as set forth in claim 1.
